(19) United States
(12) Patent Application Publication
(10) Pub. No.: US 2011/0090460 A1
(43) Pub. Date: Apr. 21, 2011

(54) OPHTHALMOLOGIC LENS ASSEMBLY

(75) Inventors: Raymond D. Graham, Renton, WA (US); Peter G. Harrington, Shoreline, WA (US)

(73) Assignee: OCULAR INSTRUMENTS, INC., Bellevue, WA (US)

(21) Appl. No.: 12/581,736

(22) Filed: Oct. 19, 2009

(51) Int. Cl. A61B 3/10; B29C 65/42; B29C 65/54

(52) U.S. Cl. 351/219; 156/293

(57) ABSTRACT

An ophthalmologic lens assembly includes a glass prism bonded to a meniscus lens at a posterior end of the glass prism. The glass prism is surrounded by a housing that is bonded to the meniscus lens. In one embodiment, the housing and a meniscus lens blank are processed by grinding and polishing the housing and meniscus lens blank together to form a posterior lens surface of a meniscus contact lens. In other embodiment, only the meniscus lens blank is processed by grinding and polishing to form a posterior lens surface of a meniscus contact lens.

### FIELD OF THE INVENTION

The technology disclosed herein relates generally to optical lenses and to ophthalmologic lenses with glass prisms in particular.

### BACKGROUND

To examine and treat different areas of the eye, ophthalmologists and optometrists often use specialized ophthalmologic lenses to view remote areas of the eyeball. Such lenses may include prisms having reflecting surfaces thereon that allow the user to view areas at sharp angles with respect to the front of the eye.

With most ophthalmologic lenses, there are distinct advantages to having a prism made of a glass material rather than an optical plastic. For example, some glasses have lower dispersion and/or higher transmission characteristics than plastics. In addition, glass prism lenses can be coated with high performance materials that are not possible to adhere to plastic lenses due to the high temperatures required to apply the materials.

To maximize the brightness of a prism, it is known to design the reflecting surfaces to reflect light using total internal reflection (TIR). However any prism that uses TIR surfaces is also susceptible to TIR failure when something touches the exterior of a reflecting surface or the exterior reflecting surface is exposed to a liquid or other substance that changes the index of refraction of the surface. To avoid this, ophthalmologic lenses with plastic prisms and TIR reflecting surfaces are often placed in a plastic housing to prevent TIR failure. However such lenses also suffer from the drawbacks associated with plastic prisms. On the other hand, lens designs with glass prisms are not easily placed in a housing due to difficulties in getting a housing material to adhere to the glass and because of the differing grinding and polishing characteristics of the glass and housing materials. Therefore, the reflecting surfaces of most lens assemblies with glass prisms are coated with a minor material such as aluminum. The minor coating eliminates the possibility of TIR failure but also reduces the brightness the lens. In addition, a mirror coating can limit the use of the lens assembly. For example, some mirrored lenses cannot be used with certain types of lasers.

Given these problems, there is a need for an ophthalmologic lens design that incorporates a glass prism having TIR reflecting surfaces but is not subject to TIR failure.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

To address the above described problems and others, the technology described herein is an ophthalmologic lens assembly having a glass prism with an anterior lens surface and a posterior lens surface. A meniscus lens is bonded to the posterior lens surface of the prism. A housing surrounds the glass prism to protect and seal the prism and any internal reflecting surfaces. In one embodiment, the housing is made of the same or similar material as the meniscus lens.

In another aspect of the disclosed technology, an ophthalmologic lens assembly is made by bonding a finished anterior lens surface of a meniscus lens blank to a posterior lens surface of a glass prism. The glass prism and meniscus lens blank are inserted into a housing blank and secured together. The housing blank and the meniscus lens blank are then processed to form a posterior lens surface of a meniscus contact lens.

In another embodiment, the ophthalmologic lens assembly is made by first securing a finished anterior lens surface of a meniscus lens blank to a housing blank. A posterior lens surface of a glass prism is then bonded to the finished anterior lens surface of the meniscus lens blank. The posterior end of the meniscus lens blank is then processed to form a posterior lens surface of a meniscus contact lens.

In yet another embodiment, the housing blank and meniscus lens blank are mechanically secured with threads or the like, The meniscus lens blank alone or the meniscus lens blank and the housing blank together are processed to form a meniscus contact lens.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a cross section of an ophthalmologic lens assembly constructed in accordance with a first embodiment of the disclosed technology;

FIG. 2 illustrates a cross section of an ophthalmologic lens assembly constructed in accordance with another embodiment of the disclosed technology;

FIG. 3 illustrates a cross section of an ophthalmologic lens assembly constructed in accordance with another embodiment of the disclosed technology;

FIG. 4 illustrates a cross section of an ophthalmologic lens assembly constructed in accordance with another embodiment of the disclosed technology; and

FIGS. 5 and 6 illustrate cross sections of an ophthalmologic lens assembly constructed in accordance with other embodiments of the disclosed technology.

### DETAILED DESCRIPTION

As will be set forth in detail below, the technology described herein relates to an ophthalmologic lens assembly that incorporates a glass prism that may have one or more reflecting surfaces that use TIR. To protect the prism and the reflecting surfaces of the prism, the glass prism is surrounded by a housing, which is secured and sealed to a meniscus lens. In some embodiments, the meniscus lens is made of the same or a similar material as the housing such that the housing and meniscus lens can be bonded together in order to seal the glass prism in the housing.

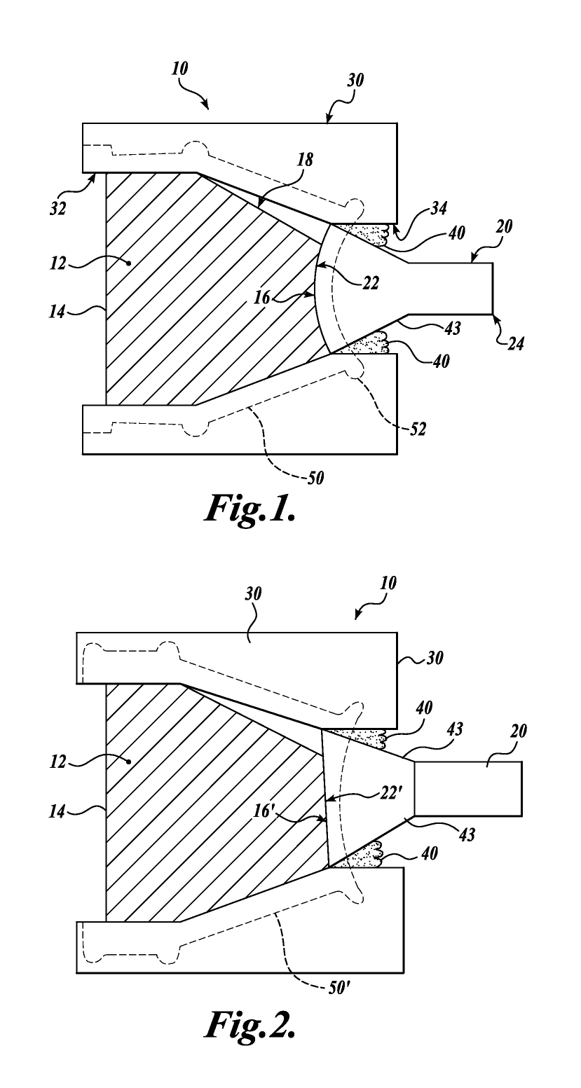

As shown in FIG. 1, an ophthalmologic lens assembly 10 includes a glass prism 12 having an anterior lens surface 14 and a posterior lens surface 16. In some embodiments, the glass prism includes one or more reflecting surfaces 18 cut or formed into the glass prism 12 that reflect light by total internal reflection. In one embodiment, the glass prism 12 is made of quartz. However, other optical glasses could be used.

A meniscus lens blank 20 has a finished anterior lens surface 22 that is shaped to match the posterior lens surface 16 of the glass prism 12 such that the anterior lens surface 22 can be bonded to the posterior lens surface 16. In one embodiment, the anterior lens surface 22 of the meniscus lens blank 20 is bonded to the posterior lens surface 16 of the glass prism 12 with an optically matched adhesive or the like. Depending on the position and number of reflecting surfaces on the glass prism, the posterior lens surface 16 of the glass prism 12 may not be circular in cross section where it is bonded to the anterior lens surface 22 of the meniscus lens blank. In one embodiment, the meniscus lens blank 20 has a circular cross section where it is bonded to the posterior lens surface 16 of the glass prism 12. The meniscus lens blank 20 therefore serves as a connecting element between the often non-circular posterior end of the glass prism and a circular meniscus contact lens that will contact a patient's eye. In the embodiment shown, the meniscus lens blank 20 has a post 24 at its posterior end that is removed in later processing.

After cleaning any excess adhesive on the glass prism 12 and the meniscus lens blank 20 as a result of bonding the meniscus lens blank 20 and the glass prism 12, the joined meniscus lens blank 20 and glass prism 12 are fitted into an internal bore of a housing blank 30. In one embodiment, the internal bore of the housing blank 30 is tapered to match the contour of the glass prism 12. In the embodiment shown, the internal bore has a first diameter 32 at the anterior end that is just larger than the outer diameter of the anterior lens surface 14 of the glass prism 12. The tapered bore then necks down in the posterior direction to a smaller diameter 34 that is just larger than the outer diameter of the anterior lens surface 22 of the meniscus lens blank 20.

Any gaps 40 between the housing blank 30 and the meniscus lens blank 20 are filled with an adhesive or potting material and cured to seal the glass prism 12 in the housing blank 30. In one embodiment, the anterior end of the glass prism 12 is bonded to the inner diameter of the housing blank 30 to seal the glass prism in the housing. In another embodiment, the seal at the anterior end 14 of the glass prism 12 can be made with a cover lens (not shown).

Once the glass prism 12 and meniscus lens blank 20 are bonded to the housing blank 30, the external surface of the housing blank can be machined to give the housing its final outer shape. The posterior end of the meniscus lens blank 20 and the housing blank 30 are processed by, for example, grinding and polishing the lens blank and the housing blank to form a posterior lens surface of a meniscus contact lens as shown by the dashed outline 50. In the embodiment shown, the outer shape of the housing blank 30 along the body of the lens assembly is machined to have a generally cylindrical section that abuts a generally conical section with diameter that tapers smaller towards the posterior end of the lens assembly. In the embodiment shown, the meniscus contact lens has a posterior lens surface with a flange 52 at its outer diameter that includes material from the housing blank 30.

As indicated above, the housing blank 30 and meniscus lens blank 20 are preferably formed of the same or similar materials that bond well together and have similar grinding and polishing characteristics. In one embodiment, both the housing blank 30 and meniscus lens blank 20 are both made of acrylic. However other biocompatible, optical polymers could also be used.

FIG. 2 shows a variation of the lens assembly 10. In this embodiment, a lens assembly 10 is like that shown in FIG. 1 but the glass prism 12 has a posterior lens surface 16′ that is planar. A corresponding anterior lens surface 22′ of the meniscus lens blank 20 is also planar and is bonded to the posterior lens surface 16′. In one embodiment, the anterior lens surface 22′ of the meniscus lens blank 20 is bonded to the posterior lens surface 16′ of the glass prism 12 with an optically matched adhesive. The housing blank 10 and the meniscus lens blank 20 are processed by machining, polishing, grinding, etc., to form a plano/concave meniscus contact lens as indicated by the dotted line 50′.

Although the anterior lens surfaces 14 of the glass prisms are shown as being planar in the embodiments shown in FIGS. 1 and 2, it will be appreciated that the shape of the anterior lens surfaces may be curved if desired. In addition, in the embodiments illustrated in FIGS. 1 and 2, the meniscus lens blank 20 has a tapered outer diameter 43 that meets the housing blank. However, it will be appreciated that the meniscus lens blank may be more cylindrical with a minimal gap that can be filled with an adhesive to bond the meniscus lens blank to the housing blank.

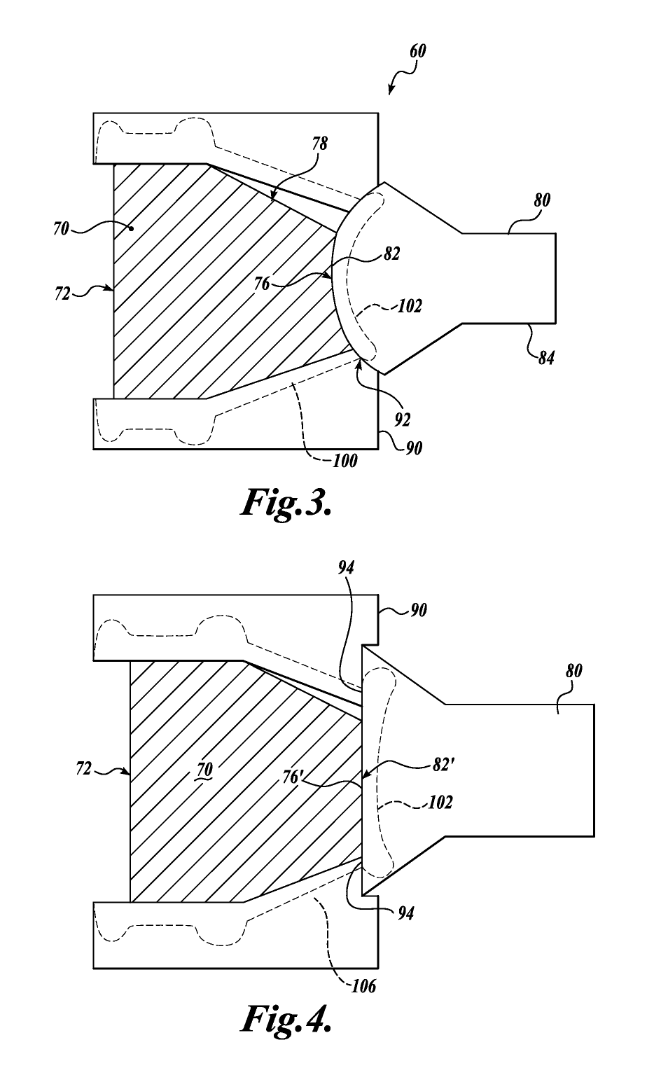

FIG. 3 illustrates another embodiment of a lens assembly in accordance with the disclosed technology. In this embodiment, a lens assembly 60 includes a glass prism 70 having an anterior lens surface 72, a posterior lens surface 76 and may include one or more reflecting surfaces 78. The reflecting surfaces 78 are designed to reflect by total internal reflection.

A meniscus lens blank 80 has a finished anterior lens surface 82 that is shaped to match the shape of the posterior lens surface 76 of the glass prism 70 such that the two lens surfaces can be optically bonded together. The meniscus lens blank 80 has a post 84 at its posterior end that facilitates handling and that will be removed in later processing.

Surrounding the glass prism 70 is a housing blank 90. In this embodiment, the posterior end of the housing blank 90 is countersunk with a concave recess 92 having a curvature that is shaped to match the curvature of the finished anterior lens surface 82 of the meniscus lens blank 80. The diameter of the meniscus lens blank 80 at the finished anterior lens surface 82 is selected such that at least a portion of the anterior lens surface engages the recess 92 so that the housing blank 90 and the meniscus lens blank 80 can be bonded together. Once the housing blank 90 and the meniscus lens blank 80 are bonded together, the glass prism 70 is inserted into a central hollow bore of the housing blank 90. The posterior lens surface 76 of the glass prism 70 is then bonded to the finished anterior lens surface 82 of the meniscus lens blank 80 with a suitable optically matched adhesive. Care should be taken to avoid getting any adhesive on the reflecting surfaces 78 of the glass prism.

Once the glass prism is inserted into the housing blank 90 and bonded to the finished anterior lens surface 82 of the meniscus lens blank 80, the outer surface of the housing blank 90 can be machined to give the housing its final outer shape as indicated by the dotted outline 100. In the embodiment shown, the final outer shape has a generally cylindrical section that abuts a conical section with an outer diameter that tapers downwards towards the meniscus contact lens. The posterior end of the meniscus lens blank 80 is also processed by grinding and polishing to form a posterior lens surface of a meniscus contact lens. In the embodiment shown in FIG. 3, the flange on the meniscus contact lens is only made of material from the meniscus lens blank 80.

FIG. 4 shows a variation of the lens assembly 60 shown in FIG. 3. In this embodiment, a posterior lens surface 76′ of the glass prism 70 is planar. The corresponding finished anterior lens surface 82′ of the meniscus lens blank 80 is also planar and is bonded to the glass prism 70. The diameter of the anterior lens surface 82′ is larger than the diameter of the posterior lens surface 76′ of the glass prism so that an outer perimeter of the meniscus lens blank 80 overlaps a portion of the housing blank 90. The housing blank 90 and the meniscus lens blank 80 are bonded along a generally flat circular recess 94 at the posterior end of the housing blank 90. After bonding the meniscus lens blank 80 to the housing blank 90, the housing blank can be machined or processed to give the housing its desired external shape as indicated by the dotted line 106. The meniscus lens blank 80 is also processed by grinding and polishing for example, the posterior end of the lens blank to create a posterior lens surface of a plano/concave meniscus contact lens. Although the anterior lens surfaces of the glass prisms 70 are shown as being planar in the embodiments shown in FIGS. 3 and 4, it will be appreciated that the anterior lens surfaces may also be curved if desired.

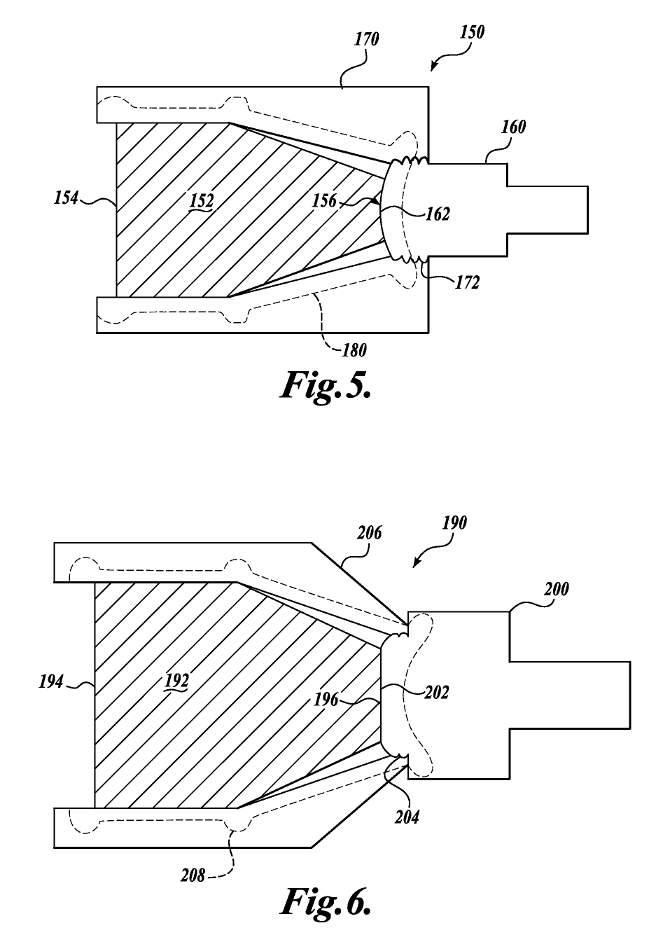

FIGS. 5 and 6 illustrate other embodiments of lens assemblies in accordance with the disclosed technology. In the embodiment shown in FIG. 5, a lens assembly 150 has a glass prism 152 with an anterior lens surface 154 and a posterior lens surface 156. A meniscus lens blank 160 has a finished anterior lens surface 162 that is bonded to the posterior lens surface 156 of the glass prism 152. A housing blank 170 is mechanically secured to the meniscus lens blank 160 with a number of cooperating threads 172 formed on the meniscus lens blank 160 and the housing blank 170. The housing blank 170 may be secured and sealed to the meniscus lens blank 160 using only the threads 172 or using the threads 172 and an adhesive. Once the housing blank 170 is secured to the meniscus lens blank 160, the housing blank 170 and the meniscus lens blank 160 are processed to shape the outer surface of the lens assembly and the posterior lens surface of the meniscus contact lens as indicated by the dotted line 180. In this embodiment, an outer flange of the contact meniscus lens may include material from the housing blank 170. Therefore it is preferable that the meniscus lens blank 160 and the housing blank 170 have similar processing characteristics.

FIG. 6 shows a similar lens assembly to that shown in FIG. 5. In this embodiment, a lens assembly 190 has a glass prism 192 with an anterior lens surface 194 and a posterior lens surface 196. A meniscus lens blank 200 has a finished anterior lens surface 202 that is bonded to the posterior lens surface 196 of the glass prism. In addition, the meniscus lens blank 200 includes a number of threads 204 that engage a corresponding set of threads on the housing blank 206 to secure and seal the housing to the meniscus lens. The housing blank 206 and the meniscus lens blank 200 are then processed to shape the outer surface of the lens assembly and to form the posterior lens surface of the meniscus contact lens as indicated by the dotted line 208. In this embodiment, the outer flange of the posterior lens surface of the contact meniscus lens does not include material from the housing blank 206. Therefore, the housing blank 206 the meniscus lens blank 200 can be made of materials having different processing characteristics. In one embodiment, the housing blank 206 can be made of aluminum and the meniscus lens blank 200 can be made of acrylic.

The glass prisms in the embodiments shown in FIGS. 1-6 may have anterior lens surfaces or other surfaces that are coated with high performance materials such as a non-reflective lens coatings. Such coatings are preferably applied prior to bonding the glass prism to the meniscus lens blank. In addition, the lens assemblies may include a removable protective lens (not shown) that is positioned over the anterior lens surface of the glass prism to protect the anterior lens surface of the glass prism.

While illustrative embodiments have been illustrated and described, it will be appreciated that various changes can be made therein without departing from the scope of the invention. For example, in some embodiments, it may be desirable to coat one or more reflecting surfaces of a glass prism with a mirror material but to still place the mirrored prism in a housing. In addition, although the disclosed embodiments show that the housing blanks and meniscus lens blanks are bonded with adhesives or mechanically secured with threads, it will be appreciated that there are other ways of securing these parts together including welding or fusing for example. In addition, the housing blanks in the disclosed embodiments are illustrated as being made of a single piece into which the glass prism is secured. However, it is possible to form the housing blank using two or more pieces that are fitted around the glass prism. This is useful where the shape of the prism and meniscus lens assembly do not easily permit the glass prism to be fitted within a bore of a housing blank. Therefore, the scope of the invention is to be determined from the following claims and equivalents thereof.

The embodiments of the invention in which an exclusive property or privilege is claimed are defined as follows:

1. An ophthalmologic lens assembly, comprising:
a glass prism having a posterior lens surface and an anterior lens surface;
a meniscus lens having an anterior lens surface bonded to the posterior lens surface of the glass prism and a posterior lens surface that is adapted to contact a patient's eye;
a housing having a central hollow bore in which the glass prism is fitted and a posterior end which secured to the meniscus lens.

2. The ophthalmologic lens assembly of claim 1, wherein the glass prism includes one or more reflecting surfaces that reflect by total internal reflection.

3. The ophthalmologic lens assembly of claim 2, wherein the one or more reflecting surfaces extend to the posterior lens surface of the glass prism such that the posterior lens surface of the glass prism is not circularly symmetric.

4. The ophthalmologic lens assembly of claim 3, wherein the anterior lens surface of the meniscus lens is circularly symmetric and is larger in diameter than the posterior lens surface of the glass prism.

5. The ophthalmologic lens assembly of claim 1, wherein the posterior lens surface of the meniscus lens has an outer flange that includes material from the housing.

6. The ophthalmologic lens assembly of claim 1, wherein the housing is bonded to the meniscus lens at the anterior lens surface of the meniscus lens.

7. The ophthalmologic lens assembly of claim 1, wherein the housing is secured to the meniscus lens with one or more threads.

8. The ophthalmologic lens assembly of claim 1, wherein the housing and the meniscus lens are made of material having substantially similar grinding and polishing characteristics.

9. The ophthalmologic lens assembly of claim 1, wherein the housing and the meniscus lens are made of the same material.

10. The ophthalmologic lens assembly of claim 9, wherein the material is acrylic.

11. A method of assembling an ophthalmologic lens assembly, comprising:
bonding an anterior lens surface of a meniscus lens blank to a posterior lens surface of a glass prism;
positioning the glass prism and the meniscus lens blank into a hollow bore of a housing blank;
securing the meniscus lens blank and the housing blank together at a posterior end of the housing blank; and
processing the posterior end of the housing blank and the posterior end of the meniscus lens blank to form a posterior lens surface of a meniscus contact lens.

12. The method of claim 11, wherein securing the meniscus lens blank and the housing blank together includes adhesively securing the meniscus lens blank and the housing blank together.

13. The method of claim 11, wherein securing the meniscus lens blank and the housing blank together includes mechanically securing the meniscus lens blank and the housing blank together.

14. The method of claim 11, wherein the posterior end of the housing blank and the posterior end of the meniscus lens blank are processed by simultaneously grinding or machining the posterior end of the housing blank and the posterior end of the meniscus lens blank.

15. The method of claim 14 wherein the posterior end of the housing blank and the posterior end of the meniscus lens blank are ground such that the posterior lens surface of the meniscus contact lens has an outer flange formed of material from the housing lens blank.

16. A method of assembling an ophthalmologic lens assembly, comprising:
securing an anterior lens surface of a meniscus lens blank to a corresponding mating surface on a housing blank;
positioning a glass prism within a hollow bore of the housing blank and bonding a posterior lens surface of the glass prism to the anterior lens surface of the meniscus lens blank; and
processing a posterior end of the meniscus lens blank to form a posterior lens surface of a meniscus contact lens.

17. The method of claim 16, wherein securing the anterior lens surface of the meniscus lens blank to the corresponding mating surface on the housing blank includes adhesively securing the anterior lens surface of the meniscus lens blank to the mating surface on the housing blank.

18. The method of claim 16, wherein securing the anterior lens surface of the meniscus lens blank to the corresponding mating surface on the housing blank includes mechanically securing the anterior lens surface of the meniscus lens blank to the mating surface on the housing blank.
